(19) United States
(12) Patent Application Publication
(10) Pub. No.: US 2014/0316252 A1
(43) Pub. Date: Oct. 23, 2014

(54) MARKER AND METHOD OF ESTIMATING SURGICAL INSTRUMENT POSE USING THE SAME

(71) Applicant: SAMSUNG ELECTRONICS CO., LTD., Suwon-si (KR)

(72) Inventors: No San KWAK, Suwon-si (KR); Seung Yong HYUNG, Yongin-si (KR); Woong KWON, Seongnam-si (KR); Suk June YOON, Seoul (KR)

(73) Assignee: SAMSUNG ELECTRONICS CO., LTD., Suwon-si (KR)

(21) Appl. No.: 14/151,268

(22) Filed: Jan. 9, 2014

(30) Foreign Application Priority Data
Apr. 23, 2013 (KR) 10-2013-0044672

(51) Int. Cl. A61B 5/06

(52) U.S. Cl. CPC A61B 5/064
USPC 600/424

(57) ABSTRACT

A marker includes a basal surface, and a plurality of reference lines provided at the basal surface in a longitudinal direction of the basal surface. The reference lines may have different gradients. The marker may be attached to an instrument and a camera may capture an image of the marker. Pose information of the instrument may be estimated based on the captured image.

### CROSS-REFERENCE TO RELATED APPLICATIONS

This application claims the priority benefit of Korean Patent Applications No. 10-2013-0044672, filed on Apr. 23, 2013 in the Korean Intellectual Property Office, the disclosure of which is incorporated herein by reference.

### BACKGROUND

1. Field

Embodiments disclosed herein relate to a marker shaped to facilitate detection of rotation information and a method of estimating a surgical instrument pose using the same.

2. Description of the Related Art

Minimally invasive surgery refers to surgical methods which may minimize the size of an incision. Different from laparotomy which uses a relatively large surgical incision through a part of a human body (e.g., the abdomen), in minimally invasive surgery, after forming one or more small ports (or incisions) of about 0.5 cm to about 1.5 cm through the abdominal wall, an operator inserts an endoscope and a variety of surgical instruments through the port, to perform surgery while viewing an image.

As compared to laparotomy, minimally invasive surgery has several advantages, such as low pain after surgery, an earlier recovery, an earlier restoration of ability to eat, a shorter hospitalization, a rapid return to daily life, and superior cosmetic effects owing to a smaller incision. Accordingly, minimally invasive surgery has been used in various procedures including gall resection, prostate cancer, and herniotomy operations, etc, and the use range thereof increasingly expands.

In general, a surgical robot used in minimally invasive surgery includes a master device and a slave device. The master device generates a control signal corresponding to an operator's (e.g., a doctor's) manipulation to transmit the control signal to the slave device. The slave device receives the control signal from the master device to perform manipulation required for a surgical operation to be performed on a patient. The master device and the slave device may be integrated with each other, or may be separately arranged in an operating room.

The slave device generally includes at least one robot arm, an end of which is provided with a surgical instrument. The surgical instrument is introduced into the patient's body and performs surgical motion on a surgical region inside the patient's body in response to a control signal transmitted from the master device. In minimally invasive surgery and laparotomy using surgical robots, it may be important to accurately estimate a pose of the surgical instrument introduced into the patient's body and to control the motion of the surgical instrument.

### SUMMARY

It is an aspect of the present invention to provide a marker shaped to facilitate detection of a position of a surgical instrument and rotation information and a method of estimating a surgical instrument pose using the same.

Additional aspects of the invention will be set forth in part in the description which follows and, in part, will be apparent from the description, or may be learned by practice of the invention.

In accordance with an aspect of the invention, a marker includes a basal surface, and a plurality of reference lines provided at the basal surface in a longitudinal direction of the basal surface, the reference lines having different gradients.

The plurality of reference lines may include a first reference line parallel to the longitudinal direction of the basal surface and a second reference line having a predetermined gradient with respect to the first reference line. The first reference line and the second reference line have different colors respectively. The marker may have one end and another end in a longitudinal direction thereof, and a distance between the first reference line and the second reference line may increase from one end to the other end of the marker. The marker may have a length to surround a part of the periphery of a surgical instrument.

In accordance with another aspect of the invention, a method of estimating a surgical instrument pose using a marker is disclosed. For example, the marker may include a basal surface and a plurality of reference lines provided at the basal surface in a longitudinal direction of the basal surface, and the reference lines may have different gradients. The method may include detecting the marker from an image acquired via a camera, and estimating a pose of a surgical instrument using the detected marker.

The marker may be detected by extracting a region corresponding to the basal surface from the image acquired via the camera, removing noise from the extracted region, detecting the rim of the region from which noise has been removed, judging whether or not the detected rim of the region has the same shape as the rim of a preset marker, and acquiring a color image of the region if the rim of the region has the same shape as the rim of the preset marker.

The extracting of the region corresponding to the basal surface may be performed by: converting the image acquired via the camera into a black-and-white image and subjecting the converted black-and-white image to binarization on the basis of a color-brightness of the basal surface.

The removal of the noise from the extracted region may be implemented by filling an empty portion within the region via a closing operation of the binary image.

Extraction of the region corresponding to the basal surface from the image acquired via the camera may be repeated if the rim of the region does not have the same shape as the rim of the preset marker.

After acquisition of the color image of the region, it may be judged whether or not the plurality of reference lines is present in the acquired color image, and the color image may be used as the marker if the plurality of reference lines is present in the color image.

Estimating of the pose of the surgical instrument may be performed by: acquiring position information on each apex of the detected marker, marking the marker according to the position information in an X-Y plane, extracting the plurality of reference lines from the marker, estimating a position of the surgical instrument using a relationship between the plurality of extracted reference lines and the X-axis of the X-Y plane, and estimating the pose of the surgical instrument using a length ratio of two sides of the marker parallel to the Y-axis of the X-Y plane as well as the relationship between the plurality of extracted reference lines and the X-axis of the X-Y plane.

Estimation of the position of the surgical instrument may include calculating a half line that originates from the center of one reference line and passes through the center of the other reference line among the plurality of extracted reference lines, selecting a reference apex of the marker using an angle between the calculated half line and the X-axis, matching the marker to a preset real marker using the selected reference apex, and estimating the position of the surgical instrument using the position information on each apex of the marker and identification information on the preset real marker. Upon selection of the reference apex of the marker, one apex, located closest to the reference line from which the half line originates, among apexes having positive values when substituted into a preset linear equation is selected as the reference apex if the angle between the half line and the X-axis is 180 degrees or more. Alternatively, one apex, located closest to the reference line from which the half line originates, among apexes having negative values when substituted into a preset linear equation is selected as the reference apex if the angle between the half line and the X-axis is less than 180 degrees.

Estimating of the pose of the surgical instrument may be implemented by calculating a roll-direction rotation angle, a yaw-direction rotation angle, and a pitch-direction rotation angle of the marker. The roll-direction rotation angle of the marker may be calculated using a distance between the centers of the plurality of reference lines. The yaw-direction rotation angle of the marker may be calculated using an angle between the X-axis and a half line that originates from the center of one reference line and passes through the center of the other reference line among the plurality of reference lines. The pitch-direction rotation angle of the marker may be calculated using the length ratio of the two sides parallel to the Y-axis of the X-Y plane. Assuming that one of the two sides closest to the Y-axis is a first side and the other side farther from the Y-axis is a second side, the pitch-direction rotation angle of the marker may have a positive value if the length of the first side is less than the length of the second side, and the pitch-direction rotation angle of the marker may have a negative value if the length of the first side is greater than the length of the second side. The calculating of the roll-direction rotation angle and the pitch-direction rotation angle of the marker may be performed by reference to a lookup table.

In accordance with another aspect of the invention, a surgical robot includes a master device to generate and transmit a control signal and a slave device to receive the control signal and to operate at least one robot arm by controlling respective joints of the at least one robot arm based on the received control signal. The slave device may include a camera disposed at an end of a first robot arm, and an instrument disposed at an end of a second robot arm. A marker may be attached to the instrument, and the marker may include a basal surface and a plurality of reference lines provided at the basal surface in a longitudinal direction of the basal surface, the reference lines having different gradients.

The basal surface may have a substantially rectangular shape and be wound around a portion of the instrument. The camera may be configured to, suitable for, capable of, or adapted to capture an image of a region including the marker.

The surgical robot may include at least one processor to detect the marker in the captured image by extracting a region corresponding to the basal surface from the image, removing noise from the extracted region, detecting a rim of the noise-removed region, and acquiring a color image of the extracted region if the detected rim has a same shape as a preset rim of the marker. The at least one processor may detect the marker in an acquired color image if it is determined the plurality of reference lines exist in the acquired color image.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a view showing an outer appearance of a surgical robot;

FIG. 2 is a view showing one example of the shape of a marker attached to a surgical instrument;

FIG. 3 is a flowchart schematically showing a method of estimating a surgical instrument pose using the marker of FIG. 2;

FIG. 4 is a flowchart showing operation S310 of FIG. 3 in detail;

FIG. 5 is a flowchart showing operation S320 of FIG. 3 in detail;

FIG. 6 is a view showing an image of the surgical instrument acquired via a camera;

FIG. 7 is a view showing an image of FIG. 6 subjected to binarization on the basis of the basal surface of the marker;

FIG. 8 is a view showing an image of FIG. 7 subjected to closing operation;

FIGS. 9 and 10 are views showing selection of a reference apex of the marker;

FIG. 11 is a view showing calculation of a roll-direction rotation angle of the marker;

FIG. 12 is a view showing calculation of a yaw-direction rotation angle of the marker; and

FIG. 13 is a view showing calculation of a pitch-direction rotation angle of the marker.

### DETAILED DESCRIPTION

Aspects, specific advantages and novel features of the embodiments of the present invention will become apparent with reference to the following detailed description and embodiments described below in detail in conjunction with the accompanying drawings. It is noted that the same or similar elements are denoted by the same reference numerals even though they are depicted in different drawings. In addition, a detailed description of well-known techniques may be omitted to avoid unnecessarily obscuring the present disclosure. Herein, the terms first, second, etc. are used simply to discriminate any one element from other elements, and the elements are not limited to these terms.

Hereinafter, reference will now be made in detail to the embodiments of the present invention, examples of which are illustrated in the accompanying drawings, wherein like reference numerals refer to like elements throughout.

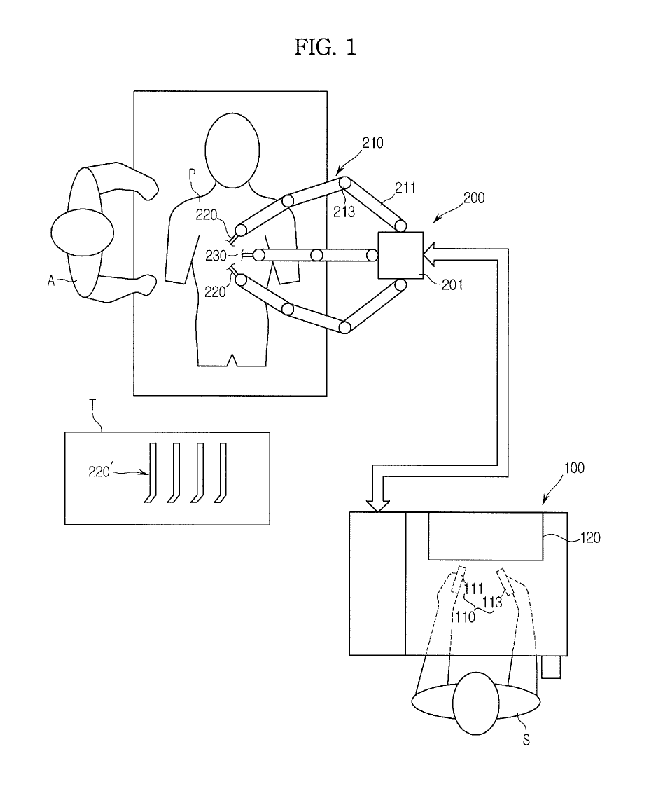

FIG. 1 is a view showing an example of an outer appearance of a surgical robot.

The surgical robot may generally include a slave device 200 to perform surgery on an object (e.g., a patient P who lies on an operating table), and a master device 100 to assist an operator (e.g., a doctor) in remotely controlling the slave device 200. As one example, and as shown in FIG. 1, at least one assistant A who assists the operator S may be located near the patient P.

Here, assisting the operator may refer to assisting a surgical operation or maneuver by the operator in a real space where the patient P is located. This assistance may include changing or switching out surgical instruments disposed at the end of a robot arm of the slave device, physically positioning a robot arm of the slave device, or performing other tasks, without being in any way limited thereto. For example, various surgical instruments may be used according to the kind of surgery and the number of robot arms 210 of the slave device 200, and consequently the number of surgical instruments used at once may be limited or finite. Accordingly, to change surgical instruments during surgery, the operator S may instruct the assistant A near the patient P to change surgical instruments, and the assistant A may remove the surgical instrument 220 from the robot arm 210 of the slave device 200 according to the operator's instruction and may mount another surgical instrument 220′ placed on a tray T to the corresponding robot arm 210.

The master device 100 and the slave device 200 may be physically separate devices without being in any way limited thereto. In one example, the master device 100 and the slave device 200 may be integrated with each other. The master device 100 and slave device 200 may communicate over a wired or wireless network, or a combination thereof.

As shown in FIG. 1, the master device 100 may include an input unit 110 and a display unit 120.

The input unit 110 may receive an instruction input by the operator, such as, for example, an instruction for selection of an operation mode of the surgical robot, or an instruction for remote control of motion of robot arms 210, surgical instruments 220, and/or an endoscope 230 of the slave device 200. The input unit 110 may include a haptic device, clutch pedal, switch, button, or the like, without being in any way limited thereto. For example, a voice recognition device, keys, joystick, keyboard, mouse, touch screen, may also be used to enable a user to control the surgical robot. It will be clearly understood that the haptic device will be described hereinafter as one example of the input unit 110, but this is one embodiment and the aforementioned various other devices may be used as the input unit 110. That is, the input unit 110 may include one or a combination of input devices as disclosed herein to control the surgical robot.

Although FIG. 1 shows the input unit 110 as including two handles 111 and 113, this is but one embodiment and the disclosure is not limited thereto. For example, the input unit 110 may include one handle, or three or more handles.

The operator S may control a motion of the robot arms 210 of the slave device 200 by moving the two handles 111 and 113 with both hands as exemplarily shown in FIG. 1. That is, if the operator S manipulates the input unit 110, a controller (not shown) of the master device 100 may generate a control signal corresponding to information regarding the state of the manipulated input unit 110 to transmit the control signal to the slave device 200.

The display unit 120 of the master device 100 may display, e.g., a real image of a surgical region inside the patient's body collected by the endoscope 230 of the slave device 200 as well as a 3D virtual image generated using medical images of the patient obtained before surgery. To this end, the master device 100 may include an image processor (not shown) that receives and processes image data transmitted from the slave device 200 to output the processed data to the display unit 120. Here, the “image data” may include a real image collected via the endoscope 230 and a 3D image generated using medical images of the patient obtained before surgery, without being in any way limited thereto.

The display unit 120 may include one or more monitors such that the respective monitors individually display information required for surgery. In one example, if the display unit 120 includes three monitors, one of the monitors may display, e.g., a real image of a surgical region collected via the endoscope 230 and a 3D image generated using medical images of the patient obtained before surgery, and the other two monitors may respectively display information regarding motion of the slave device 200 and patient information. In this case, the number of monitors may be determined in various ways according to the type or kind of information to be displayed. That is, the number of monitors may be one or may be plural. The display unit 120 may be embodied by, for example, a Liquid Crystal Display (LCD), light emitting diode (LED) display, organic light emitting diode (OLED) display, plasma display panel (PDP), cathode ray tube (CRT), and the like.

Here, “patient information” may be information regarding the state of the patient, for example, patient vital signs, such as body-temperature, pulse, respiration-rate, blood-pressure, etc. To provide the master device 100 with the vital signs, the slave device 200 that will be described hereinafter may further include a vital sign measurement unit including a body-temperature measurement module, a pulse measurement module, a respiration-rate measurement module, a blood-pressure measurement module, etc. To this end, the master device 100 may further include a signal processor (not shown) that receives and processes information transmitted from the slave device 200 to output the processed information to the display unit 120. Alternatively, or additionally, the master device may include a device (e.g., a vital sign measurement unit and/or sensors) to measure biological information regarding the operator at the master device.

The slave device 200, as exemplarily shown in FIG. 1, may include a plurality of robot arms 210, various surgical instruments 220 mounted at ends of the respective robot arms 210, and the endoscope 230.

The plurality of robot arms 210, as shown in FIG. 1 by way of example, may be coupled to a body 201 so as to be fixed to and supported by the body 201. In this case, the number of the surgical instruments 220 that are used at once as well as the number of the robot arms 210 may depend on various factors, such as diagnostic methods, surgical methods, and spatial restrictions of an operating room.

In addition, each of the plurality of robot arms 210 may include a plurality of links 211 and a plurality of joints 213. Each joint 213 may serve to connect two links 211 to each other, and may have 1 degree of freedom (DOF) or more. The DOF refers to a DOF with regard to kinematics or inverse kinematics. The DOF of a device refers to the number of independent motions of the device, or the number of variables that determine independent motions at relative positions between links. For example, an object in a 3D space defined by X-, Y-, and Z-axes has 3 DOF to determine a spatial position of the object (a position on each axis) and/or 3 DOF to determine a spatial orientation of the object (a rotation angle relative to each axis). More specifically, it will be appreciated that an object has 6 DOF if the object is movable along each of X-, Y-, and Z-axes and is rotatable about each of X-, Y-, and Z-axes.

Each joint 213 may be provided with a detector to detect information regarding the state of the joint 213. For example, the detector may include a force/torque detector to detect information regarding force/torque applied to the joint 213, a position detector to detect information regarding a position of the joint 213, and a speed detector to detect information regarding a movement speed of the joint 213. Here, the speed detector may be omitted according to the kind of a position sensor that is used as the position detector. In this case, the position sensor may be a potentiometer or an encoder, for example, without being in any way limited thereto.

The slave device 200 may include a drive unit (not shown) to control movement of the robot arm 210 in response to a control signal transmitted from the master device 100.

For example, if the operator S manipulates the input unit 110 of the master device 100, the controller (not shown) of the master device 100 generates a control signal corresponding to information regarding the state of the manipulated input unit 110 to transmit the control signal to the slave device 200. A controller (not shown) of the slave device 200 drives the drive unit (not shown) in response to the control signal transmitted from the master device 100, thereby operating the robot arm 210 by controlling the respective joints of the robot arm 210. In this case, a practical procedure of controlling rotation and movement of the robot arm 210 in a given direction based on manipulation of the input unit 110 by the operator S would be understood by one of ordinary skill in the art, and thus a detailed description thereof will be omitted herein.

Meanwhile, the respective joints of the robot arm 210 of the slave device 200, as described above, may be moved in response to the control signal transmitted from the master device 100, or may be moved by external force. That is, the assistant A located near an operating table may manually move the respective joints of the robot arm 210 to control a position and pose of the robot arm 210.

Although not shown in detail in FIG. 1, in one example, each surgical instrument 220 may include a housing mounted to the end of the robot arm 210, a shaft extending from the housing by a predetermined length, and an end effector coupled to a distal end of the shaft.

In general, the surgical instruments 220 may be basically classified into main surgical instruments and auxiliary surgical instruments. Here, the “main surgical instrument” may refer to an instrument including an end effector (e.g., a knife or a surgical needle) that performs direct surgical motion, such as, e.g., cutting, suturing, clotting, or washing, on a surgical region. The “auxiliary surgical instrument” may refer to an instrument including an end effector (e.g., a skin holder) that does not perform direct motion on a surgical region and assists motion of the main surgical instrument.

The end effector may refer to a part of the surgical instrument 220 that practically acts on a surgical region of the patient P. For example, the end effector may include a skin holder, suction line, knife, scissors, grasper, surgical needle, staple applier, needle holder, scalpel, cutting blade, etc., without being in any way limited thereto. Any other known instruments required for surgery may be used. The surgical tools or instruments may also include, for example, a, micro-dissector, tacker, suction irrigation tool, clip applier, irrigator, catheter, suction orifice, surgical knife, surgical forceps, a cautery (a tool for burning or cutting a diseased part by using electric energy or heat energy), and the like.

A drive wheel may be coupled to the housing and connected to the end effector via a wire, etc. Thus, the end effector may be operated via rotation of the drive wheel. To this end, a drive unit (not shown) to rotate the drive wheel may be provided at the end of the robot arm 210. For example, if the operator manipulates the input unit 110 of the master device 100, the master device 100 may generate a control signal corresponding to information regarding the state of the manipulated input unit 110 to transmit the control signal to the slave device 200. As the controller (not shown) of the slave device 200 drives the drive unit (not shown) in response to the control signal transmitted from the master device 100, a desired motion of the end effector may be realized. However, a mechanism to operate the end effector is not limited to the aforementioned configuration and various electrical/mechanical mechanisms may naturally be applied to realize motion of the end effector required for robotic surgery.

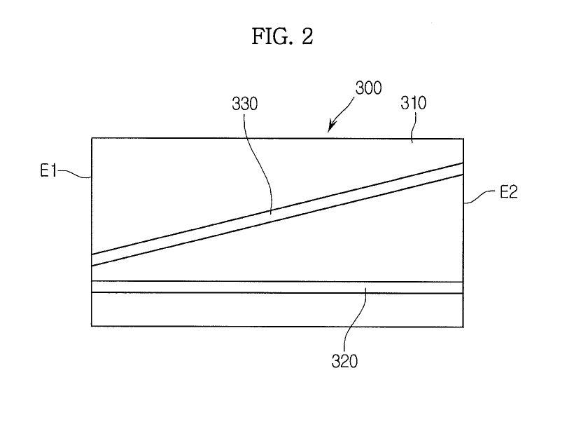

In an embodiment, a marker 300 having a shape as shown by way of example in FIG. 2, may be attached to the surgical instrument 200.

In general, in robotic surgery, a marker may be used to estimate a position and pose of the surgical instrument 220 inserted into the body of the patient P. The marker may have a specific shape. In use, the marker may be attached to the surgical instrument 220. Once the marker is detected from an image acquired via a camera, such as the endoscope 230, a position and pose of the surgical instrument 220 may be calculated using the detected shape of the marker.

The present embodiment relates to the shape of the marker having the aforementioned function. The marker according to the present embodiment may be attached to the surgical instrument 220. For example, the marker may be attached so as to be wound on the surgical instrument 220.

Referring to FIG. 2, the marker 300 according to the present embodiment may include a basal surface 310, and a plurality of reference lines 320 and 330 longitudinally formed at the basal surface 310, and the respective reference lines 320 and 330 may have different gradients. Although FIG. 2 shows two reference lines 320 and 330 formed at the basal surface 310, this is but one embodiment and a greater number of reference lines may be provided. Hereinafter, for convenience of description, the marker 300 having two reference lines, more particularly a first reference line 320 and a second reference line 330 formed at the basal surface 310 will be described by way of example.

The basal surface 310 of the marker 300 according to the present embodiment, as exemplarily shown in FIG. 2, may have a rectangular shape, without being in any way limited thereto. In addition, although the first reference line 320 may be parallel to a longitudinal direction of the basal surface 310 and the second reference line 330 may have a predetermined gradient with respect to the first reference line 320, the disclosure is not in any way limited thereto. Note that the first reference line 320 and the second reference line 330 may be non-parallel to facilitate detection of a roll-direction rotation angle of the surgical instrument 220. That is, the first reference line 320 and the second reference line 330 may eventually intersect with one another. However, the intersection need not be formed on the marker. Since rotation of the surgical instrument 220 in the roll direction changes a distance between the center of the first reference line 320 and the center of the second reference line 330 of the marker 300 detected from the image that is acquired via the camera, a rotation corresponding to the changed distance may be easily detected. Here, the “longitudinal direction” may refer to a direction parallel to a longer side among four sides of the rectangular basal surface 310.

In the present embodiment, the basal surface 310, the first reference line 320, and the second reference line 330 of the marker 300 may have different colors. This ensures easy distinction between the basal surface 310, the first reference line 320, and the second reference line 330 of the marker 300 detected from the image acquired via the camera. However, the disclosure is not so limited. Other methods may be applied to distinguish the basal surface 310, the first reference line 320, and the second reference line 330 of the marker 300. For example, different patterns may be used to indicate or represent the reference lines (e.g., dashed lines, dots, symbols, or other geometric shapes may be used to form each reference line, and each reference line may be distinguished from one another by the different respective patterns corresponding to each line.

The marker 300 according to the present embodiment, as exemplarily shown in FIG. 2, may have one end E1 and the other end E2 in a longitudinal direction. Here, the “longitudinal direction” may refer to a direction parallel to a longer side among four sides of the basal surface 310, and “one end” may refer to one longitudinal distal end and “the other end” may refer to the other longitudinal distal end.

In the marker 300 having one end E1 and the other end E2, the distance between the first reference line 320 and the second reference line 330 may increase with increasing distance from one end E1 to the other end E2, with increasing distance toward the other end E2, without being in any way limited thereto. That is, the distance between the second reference line 330 and the first reference line 320 increases from E1 to E2. Alternatively, the distance between the first reference line 320 and the second reference line 330 may decrease with increasing distance from one end E1. That is, the distance between the second reference line 330 and the first reference line 320 may decrease from E1 to E2.

In addition, the marker 300 according to the present embodiment may have a length to surround a part of the periphery of the surgical instrument 220. That is, the marker 300 according to the present embodiment may be attached to the periphery of the surgical instrument 220 so as to be wound on the surgical instrument 220. As described above, to facilitate detection of the roll-direction rotation angle of the surgical instrument 220, the first reference line 320 and the second reference line 330 of the marker 300 may be formed in such a way that a distance therebetween increases from one end E1 to the other end E2. However, if one end E1 and the other end E2 of the marker 300 meet each other as the marker 300 is rolled, the distance between the first reference line 320 and the second reference line 330 may suddenly vary, which makes it impossible to detect the roll-direction rotation angle. Accordingly, the marker 300 according to the present embodiment may have an insufficient length to completely surround the periphery of the surgical instrument 220. For example, the marker 300 may be configured, adapted to, capable of, suitable for, or manufactured such that a length of the marker (a distance from end E1 to end E2) is less than an outer circumference or outer perimeter of the surgical instrument 220, to thereby prevent the ends E1 and E2 from meeting each other when the marker 300 is attached to the surgical instrument 220.

Hereinafter, a method of estimating a surgical instrument pose using the marker 300 according to an embodiment will be described.

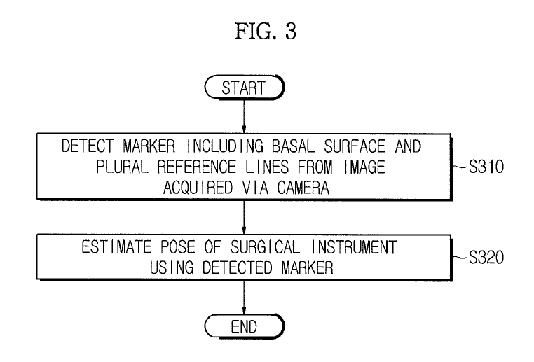

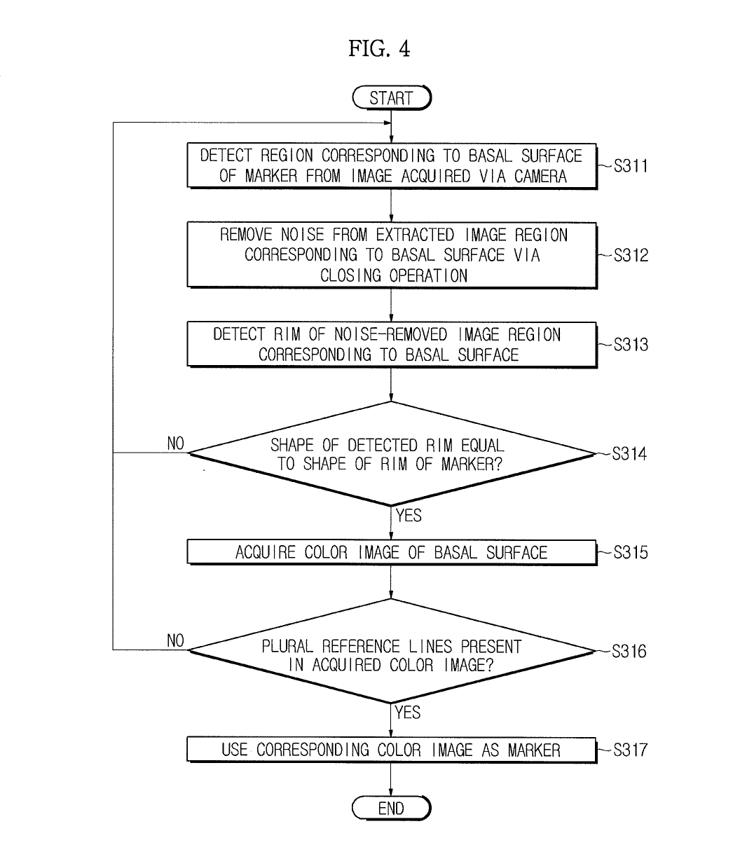

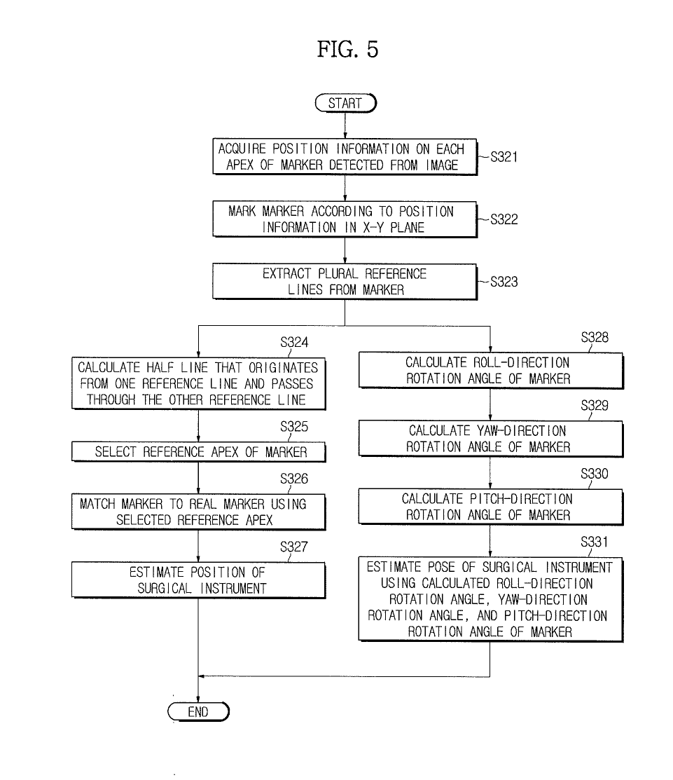

FIG. 3 is a flowchart schematically showing a method of estimating a surgical instrument pose using the marker of FIG. 2, FIG. 4 is a flowchart showing operation S310 of FIG. 3 in detail, and FIG. 5 is a flowchart showing operation S320 of FIG. 3 in detail. As disclosed above, the master device 100 may include an image processor (not shown) that receives and processes image data transmitted from the slave device 200 to output the processed data to the display unit 120. Here, the “image data” may include a real image collected via the endoscope 230 and the image processor, the controller, or the image processor together with the controller of the master device, may perform one or more of the operations disclosed herein with respect to FIGS. 3 through 5. Alternatively, an image processor and controller may be disposed in the slave device, and one or more of the operations disclosed herein with respect to FIGS. 3 through 5 may also be performed by the slave device, or some operations may be performed by a processor in the slave device while others are performed by a processor in the master device.

Referring to FIG. 3, the method of estimating a surgical instrument pose using the marker 300 according to the present embodiment may include detecting the marker 300 from an image acquired via the camera (S310) and estimating a pose of the surgical instrument 220 using the plurality of reference lines 320 and 330 included in the detected marker 300. The aforementioned endoscope 230 of the slave device 200 may be used as the “camera”, without being in any way limited thereto. An endoscope may generally also be considered as a surgical instrument and may include various surgical endoscopes, such as a thoracoscope, an arthroscope, a rhinoscope, a cysotoscope, a rectoscope, a duodenoscope, and a cardioscope, in addition to a laparoscope that is generally used in robotic surgery.

Operation S310 of detecting the marker 300 will hereinafter be described in detail.

First, referring to FIG. 4, a region corresponding to the basal surface 310 of the marker 300 may be extracted from the image acquired via the camera (S311). Here, the “image” may be a color image of a surgical region inside the patient's body.

In the present embodiment, extraction of the image region corresponding to the basal surface 310 of the marker 300 may be implemented by converting the acquired camera image into a black-and-white image, and subjecting the converted black-and-white image to binarization on the basis of the color of the basal surface 310. Here, binarization may refer to representing variously distributed RGB (red, green, blue) with only black and white. In general, binarization is an image processing method that converts a color RGB image into a gray-scale image, and thereafter converts the gray-scale image into an image composed of pixels having a brightness of 0 or 255, i.e. a white or black image on the basis of a specific critical value.

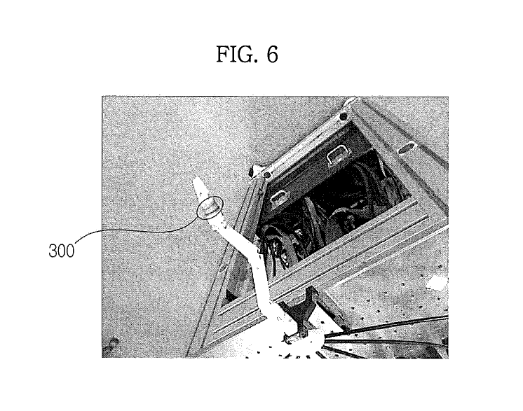

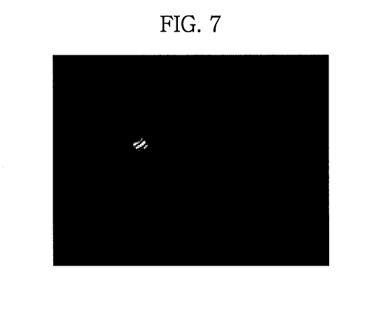

That is, a color image of the interior of the patient's body may be acquired via the camera and may be converted into a gray-scale image. The converted gray-scale image may then be subjected to binarization on the basis of a predefined color-brightness of the basal surface 310 of the marker 300 such that an image region corresponding to the basal surface 310 has a different color-brightness from that of the remaining image region, which enables extraction of the image region corresponding to the basal surface 310. FIG. 6 shows the image acquired via the camera, and FIG. 7 shows the image of FIG. 6 subjected to binarization on the basis of the basal surface 310 of the marker 300. As shown in FIG. 7 for example, the image region corresponding to the basal surface 310 may be represented by bright (white), and the remaining image region may be represented by dark (black).

Next, the region of the binary image corresponding to the basal surface 310 may be subjected to noise removal (S312).

More specifically, in the present embodiment, the marker 300 includes the plurality of reference lines 320 and 330 formed at the basal surface 310, and a color of the reference lines 320 and 330 may be different from a color of the basal surface 310. As such, as exemplarily shown in FIG. 7, in the binary image, a portion corresponding to the plurality of reference lines 320 and 330 may be represented by dark (e.g., black), differently from the image region corresponding to the basal surface 310 (e.g., white).

To accurately detect the marker 300 from the image acquired via the camera, it may be necessary for the region of the binary image corresponding to the basal surface 310 to be clearly distinguished from the surroundings. Thus, brightness of the dark portion included in the binary image region corresponding to the basal surface 310 shown in FIG. 7 may need to be enhanced.

To this end, in the present embodiment, morphology may be used, without being in any way limited thereto. In general, morphology may refer to an image processing method to change the shape of a specific object present in an image. Such morphology may include erosion operation of expanding the background and contracting the object, dilation operation of contracting the background and expanding the object, opening operation of removing fine noise, and closing operation of filling an empty space inside the object, without being in any way limited thereto.

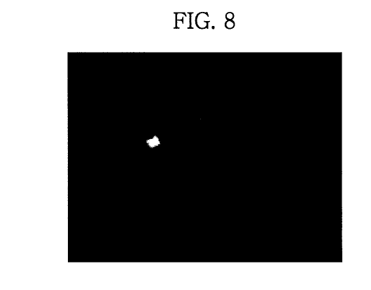

For example, in the present embodiment, an empty portion of the binary image corresponding to the plurality of reference lines 320 and 330 included in the image region corresponding to the basal surface 310 may be filled via a closing operation, although the disclosure is not in any way limited thereto. FIG. 8 shows the image in which the empty portion included in the image region corresponding to the basal surface 310 is filled via the closing operation.

Next, the rim of the noise-removed image region corresponding to the basal surface 310 may be detected (S313). Then, whether or not the detected rim of the image region corresponding to the basal surface 310 has the same shape as a preset rim of the marker 300 may be judged (S314). If the judged result shows that the rim of the image region corresponding to the basal surface 310 has the same shape as the preset rim of the marker 300, a color image of the basal surface 310 is acquired (S315). On the other hand, if the judged result shows that the rim of the image region corresponding to the basal surface 310 has a different shape from the preset rim of the marker 300, the image region is not determined to be the marker 300, and extraction of the region corresponding to the basal surface 310 from the image acquired via the camera (S311) may be repeated.

An object having color equal to or similar to that of the basal surface 310 of the marker 300 may be present in the image acquired via the camera. Thus, a plurality of objects having the same brightness as that of the basal surface 310 may be present in the binary image that is subjected to binarization on the basis of a color-brightness of the basal surface 310. In the case in which the plurality of objects having the same brightness is present in the image, to detect the marker 300, searching for an object having the same shape as the marker 300 may be necessary. Therefore, after detection of the rim of the object, whether or not the detected rim has the same shape as the marker 300 (e.g., a rectangular shape) is judged. In this case, detection of the rim of the object would be understood by those of ordinary skill in the art in the field of image processing, and thus a detailed description thereof will be omitted herein.

Next, whether or not the plurality of reference lines 320 and 330 is present in the acquired color image of the basal surface 310 is judged (S316). If the judged result shows that the plurality of reference lines 320 and 330 is present, the acquired color image of the basal surface 310 is used as the marker 300 (S317). In this case, if the judged result shows that the plurality of reference lines 320 and 330 is not present in the color image of the basal surface 310, the object is not determined to be the marker 300, and extraction of the region corresponding to the basal surface 310 from the image acquired via the camera (S311) may be repeated.

For example, the presence of the plurality of reference lines 320 and 330 in the acquired color image of the basal surface 310 may be judged by subjecting the acquired color image of the basal surface 310 to binarization on the basis of a color-brightness of the basal surface 310, or to binarization on the basis of a color-brightness of each of the plurality of reference lines 320 and 330, without being in any way limited thereto, and various other known image processing technologies may be applied.

Through implementation of the aforementioned operations, the marker 300 may be detected from the image acquired via the camera.

Thereafter, a method of estimating a pose of the surgical instrument 220 using the detected marker 300 will be described.

Referring to FIG. 5, the method of estimating a pose of the surgical instrument 220 using the marker 300 according to an embodiment includes acquiring position information on each apex of the detected marker 300 from the image acquired via the camera (S321), marking the marker 300 according to the acquired position information on each apex in an X-Y plane (S322), and extracting the plurality of reference lines 320 and 330 from the marker 300 (S323). Hereinafter, for convenience of description, the plurality of reference lines 320 and 330 will be described as including the first reference line 320 that is parallel to the longitudinal direction of the basal surface 310 and the second reference line 330 having a predetermined gradient with respect to the first reference line 320, although the number and shape of the reference lines are not limited thereto.

For example, extraction of the plurality of reference lines 320 and 330 from the marker 300 may be implemented via image processing, such as the aforementioned binarization, without being in any way limited thereto, and various other known image processing methods for object extraction may be applied.

Next, estimation of a position and pose of the surgical instrument 220 using the extracted first and second reference lines 320 and 330 may be performed. First, a method of estimating a position of the surgical instrument 220 will be described in detail.

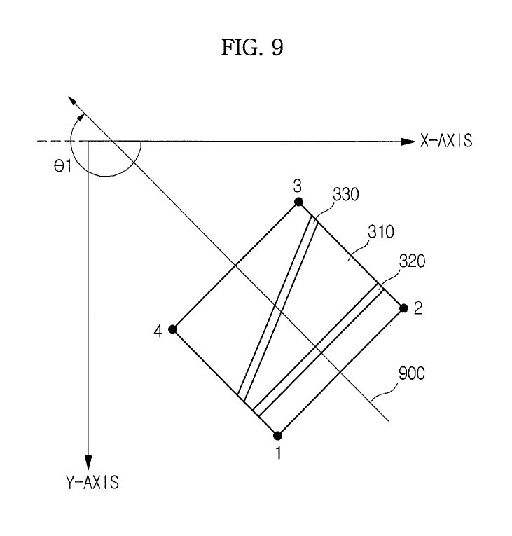

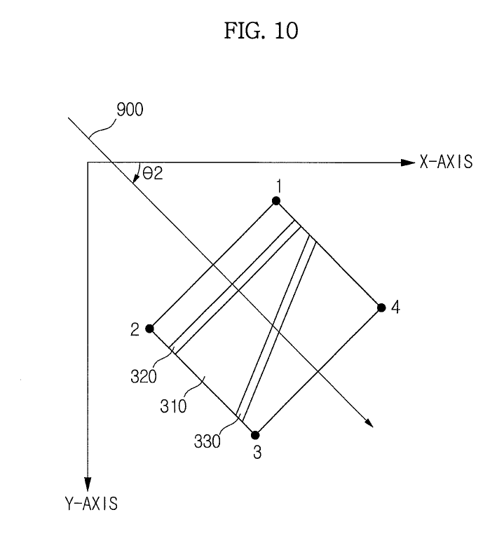

First, referring to FIG. 5, a half line passing through the center of the extracted first reference line 320 and the center of the extracted second reference line 330 may be calculated (S324). The calculated half line, designated by reference numeral 900, is shown in FIGS. 9 and 10. Here, the “half line” may refer to a straight line that originates from one point and extends in a given direction. That is, the half line may refer to a straight line that originates from the first reference line 320 and extends in a direction passing through the second reference line 330. In the present embodiment, although the half line, which originates from the first reference line 320 and extends in a direction passing through the second reference line 330, is acquired, this is but one embodiment, and an opposite direction half line that originates from the second reference line 330 and extends in a direction passing through the first reference line 320 is also possible. The direction of the half line 900 may vary according to a position of the marker 300 in image coordinates as exemplarily shown in FIGS. 9 and 10. For example, the half line may be perpendicular to the reference line from which it originates. For example, as shown in FIG. 9, the half line may be perpendicular to the first reference line 320. Alternatively, the half line may be perpendicular to the second reference line 330.

Next, a reference apex of the marker 300 may be selected using an angle between the half line calculated via operation S324 and the X-axis of the X-Y plane (S325), and the marker 300 may be matched to a real marker using the selected reference apex (S326). Here, the “reference apex” may refer to a reference point to match each apex of the marker 300 included in the image to a corresponding apex of the real marker attached to the surgical instrument 220.

Although the reference apex of the marker 300 according to the present embodiment may be one of apexes closest to the first reference line 320, this is but one embodiment, and the reference apex may be one of apexes closest to the second reference line 330.

More specifically, according to the magnitude of an angle between the half line calculated via operation S324 and the X-axis of the X-Y plane, for example, according to whether the angle between the half line and the X-axis of image coordinates is 180 degrees or more or is less than 180 degrees, an apex closest to the first reference line 320 among apexes having a positive value or negative value when substituted into a preset linear equation may be selected as the reference apex.

Explaining this with reference to FIG. 9, since an angle θ1 between the half line 900 and the X-axis is 180 degrees or more, apexes having positive values when substituted into a preset linear equation may be selected. Thus, apexes 1 and 4 may be selected. In this case, the apex 1 is closest to the first reference line 320, and therefore may be finally determined to the reference apex. Alternatively, an apex closest to the second reference line 330 may be selected. Likewise, referring to FIG. 10, since an angle θ2 between the half line 900 and the X-axis is less than 180 degrees, apexes having negative values when substituted into a preset linear equation may be selected. Thus, apexes 1 and 4 may be selected. In this case, the apex 1 is closest to the first reference line 320, and therefore may be finally determined to the reference apex. Alternatively, an apex closest to the second reference line 330 may be selected.

As a result of selecting apexes having positive values or negative values when substituted into a preset linear equation according to the angle between the half line and the X-axis of image coordinates and determining one apex closest to the first reference line 320 to the reference apex, the reference apex of the marker 300 may be detected regardless of a position of the marker 300 in the image, which facilitates matching of the marker 300 to the real marker.

Next, a position of the surgical instrument 200 may be estimated using identification information on the preset real marker as well as position information on each apex of the marker 300 (S327). Here, the “identification information on the real marker” may include information on the size of the marker, without being in any way limited thereto.

A method of estimating a pose of the surgical instrument 220 will be described. For example, the pose of the surgical instrument 220 may be estimated by calculating a roll-direction rotation angle, a yaw-direction rotation angle, and a pitch-direction rotation angle of the marker 300. Here, the “roll-direction rotation angle” may refer to a rotation angle of the surgical instrument 220 about the center axis, the “yaw-direction rotation angle” may refer to a rotation angle of the surgical instrument 220 about the center axis for leftward or rightward movement, and the “pitch-direction rotation angle” may refer to a rotation angle of the surgical instrument 220 about the center axis for upward or downward movement. In the following description, it is assumed that the center axis of the surgical instrument 220 is the X-axis of the X-Y plane. However, the disclosure is not so limited, and a different axis may be utilized as the center axis of the surgical instrument.

First, referring to FIG. 5, the roll-direction rotation angle may be calculated using a distance between the center of the first reference line 320 and the center of the second reference line 330 of the marker 300 (S328).

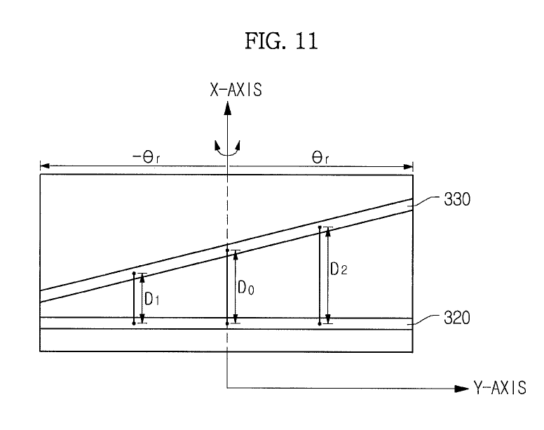

This will hereinafter be described in detail with reference to FIG. 11. For convenience of description, FIG. 11 shows an unrolled state of the marker 300 attached to the surgical instrument 220. In addition, the roll-direction rotation angle may be designated by θᵣ and −θᵣ. Here, θᵣ may refer to forward rotation, and −θᵣ may refer to reverse rotation. Alternatively, the rotation angles may refer to a clockwise or counterclockwise rotation. In addition, a distance between the center of the first reference line 320 and the center of the second reference line 330 in a state in which the roll-direction rotation angle is zero degrees is designated by D₀. If the distance between the first reference line 320 and the second reference line 330 at the center of the marker 300 detected from the image acquired via the camera is D₀, this may refer to an initial state in which the surgical instrument 220 is not yet rotated in a roll direction.

While the surgical instrument 220 is rotated about the X-axis in a roll direction, the distance between the center of the first reference line 320 and the center of the second reference line 330 of the marker 300 detected from the image acquired via the camera continuously varies from D₀ to D₁ or D₂. That is, the distance between the first reference line 320 and the second reference line 330, as described above, continuously increases from one end E1 to the other end E2 of the marker 300.

A lookup table of the roll-direction rotation angle on a per distance basis may be prepared. Thus, if the distance between the center of the first reference line 320 and the center of the second reference line 330 of the marker 300 detected from the image is measured, the roll-direction rotation angle corresponding to the measured distance may be searched for in the lookup table, which ensures easy calculation of the roll-direction rotation angle. As an example, referring to reference lines 320 and 330 as shown in FIG. 11, if a distance D₁ is measured, it may be determined that the surgical instrument has rolled in one of a clockwise or counterclockwise direction by an angle which may be determined based on the measured distance D₁. A different (e.g., opposite) result may be obtained if the distance between the second reference line 330 continuously decrease from E1 to E2. Further, an initial state (i.e., unrolled state) of the marker 300 may be determined at a position other than the center of the first reference line, and a lookup table may be generated accordingly.

Next, referring to FIG. 5, the yaw-direction rotation angle of the marker 300 may be calculated by measuring an angle between the half line 900 passing through the center of the first reference line 320 and the center of the second reference line 330 of the marker 300 and the X-axis of the X-Y plane (S329).

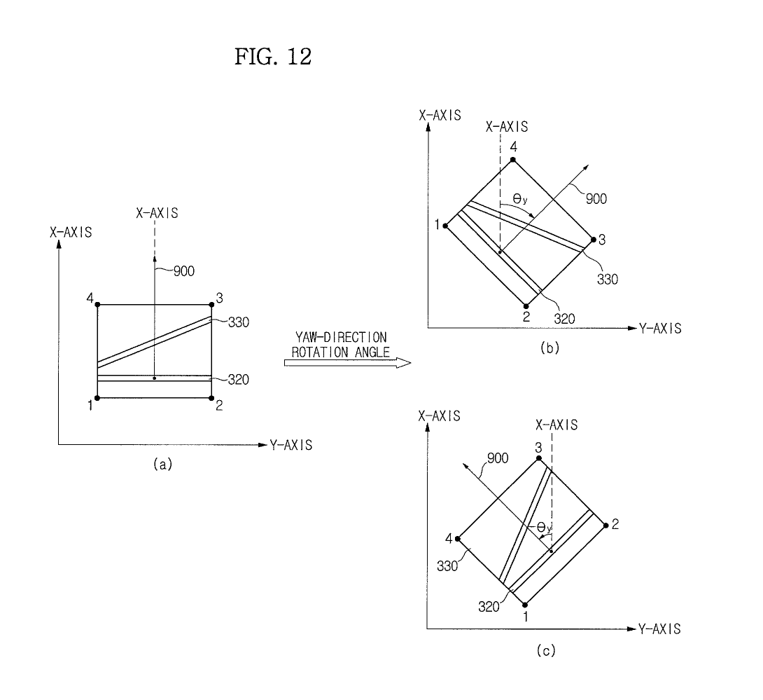

Explaining this with reference to FIG. 12, in FIG. 12(a), since an angle between the half line 900 of the marker 300 and the X-axis of the X-Y plane is zero degrees in FIG. 12(a), the yaw-direction rotation angle of the marker 300 is zero degrees, which may mean that the surgical instrument 220 is not rotated in a yaw direction. In FIG. 12(b), since the angle between the half line 900 of the marker 300 and the X-axis of the X-Y plane is θ_{y}, the yaw-direction rotation angle of the marker 300 may be θ_{y}. Likewise, in FIG. 12(c), since the angle between the half line 900 of the marker 300 and the X-axis of the X-Y plane is −θ_{y}, the yaw-direction rotation angle of the marker 300 may be −θ_{y}. Here, θ_{y} and −θ_{y} may respectively refer to a forward rotation angle and a reverse rotation angle, or clockwise rotation or counterclockwise rotation in a yaw direction, without being in any way limited thereto.

Next, referring to FIG. 5, the pitch-direction rotation angle of the marker 300 may be calculated using a ratio of two sides of the marker 300 parallel to the Y-axis of the X-Y plane (S330). Hereinafter, for convenience of description, one of the two sides of the marker 300 parallel to the Y-axis is referred to as a first side and the other side farther from the Y-axis is referred to as a second side.

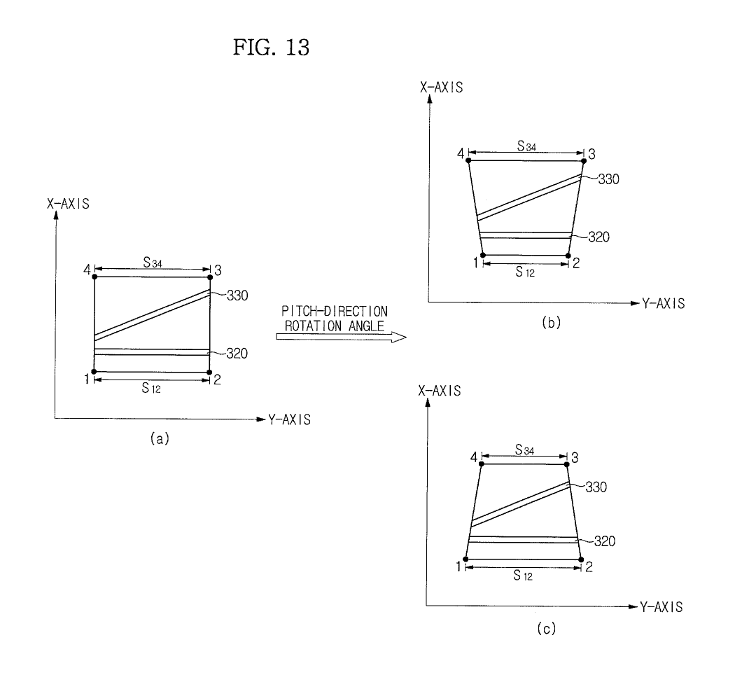

Explaining this with reference to FIG. 13, FIG. 13(a) shows a state in which the surgical instrument 220 is not moved upward or downward about the X-axis. In such a state, the first side S₁₂ and the second side S₃₄ of the marker 300 parallel to the Y-axis have the same length, and therefore a ratio of lengths of the two sides is 1. If the length ratio of the first side S₁₂ to the second side S₃₄ is 1, the pitch-direction rotation angle of the marker 300 may be zero degrees.

FIG. 13(b) shows a state in which the surgical instrument 220 is moved upward about the X-axis, and FIG. 13(c) shows a state in which the surgical instrument 220 is moved downward about the X-axis.

More specifically, in FIG. 13(b), the first side S₁₂ of the marker 300 is shorter than the second side S₃₄, which may mean or imply that a depth of the first side S₁₂ is greater than a depth of the second side S₃₄. Likewise, in FIG. 13(c), the first side S₁₂ of the marker 300 is longer than the second side S₃₄, which may mean or imply that a depth of the first side S₁₂ is less than a depth of the second side S₃₄.

In this case, as the surgical instrument 220 is rotated in a pitch direction, the length ratio of the first side S₁₂ to the second side S₃₄ varies. Thus, the pitch-direction rotation angle of the marker 300 may be calculated using the variable length ratio of the first side S₁₂ to the second side S₃₄. In this case, the pitch-direction rotation angle of the marker 300 corresponding to the length ratio of the first side S₁₂ to the second side S₃₄ may be previously given in the form of a lookup table. Thus, if the length ratio of the first side S₁₂ to the second side S₃₄ of the marker 300 detected from the image is measured, the pitch-direction rotation angle of the marker 300 corresponding to the measured length ratio of the first side S₁₂ to the second side S₃₄ may be easily acquired from the aforementioned lookup table.

Meanwhile, the length ratio of the first side S₁₂ to the second side S₃₄ upon upward movement of the surgical instrument 220 may be equal to the length ratio of the first side S₁₂ to the second side S₃₄ upon downward movement of the surgical instrument 220. Accordingly, for distinction between the two cases, the pitch-direction rotation angle of the marker 300 may be calculated to a positive value if the length of the first side S₁₂ is less than the length of the second side S₃₄, whereas the pitch-direction rotation angle of the marker 300 may be calculated to a negative value if the length of the first side S₁₂ is greater than the length of the second side S₃₄. The lookup tables for the pitch-direction rotation angle and the roll-direction rotation angle of the marker 300, or any other lookup tables which may be implemented, may be stored in a storage device. For example, the storage may be embodied as a storage medium, such as a nonvolatile memory device, such as a Read Only Memory (ROM), Programmable Read Only Memory (PROM), Erasable Programmable Read Only Memory (EPROM), and flash memory, a volatile memory device such as a Random Access Memory (RAM), a hard disc, and an optical disc, or combinations thereof. However, examples of the storage are not limited to the above description, and the storage may be realized by other various devices and structures as would be understood by those skilled in the art.

Thereafter, a pose of the surgical instrument 220 may be estimated using the roll-direction rotation angle, the yaw-direction rotation angle, and the pitch-direction rotation angle of the marker 300 calculated via the aforementioned operations S328, S329 and S330.

A minimally invasive surgical robot according to the example embodiments disclosed herein applies a marker to an instrument and estimates a pose of the instrument using the marker. Applications of the marker and estimating method are not limited to the surgical robot disclosed herein. For example, the marker and estimating method may also be applied to settings other than a medical environment. For example, the methods and marker according to the example embodiments may be utilized to perform operations in any confined space or enclosure in which an operator may need to perform controlled movements using an instrument attached to a robot arm, so as to avoid or to prevent injuries to bodies or objects, that may be located or disposed within the space or enclosure, due to imprecise movements of the robot. The methods and marker according to the example embodiments may be applied to, for example, mining operations, surveillance operations, inspection operations, repair operations, bomb disposal operations, etc., however again, the disclosure is not so limited. Further, while the operator of a surgical robot may be a doctor, the operator generally may be any user, and need not be a doctor.

The apparatus and methods according to the above-described example embodiments may use one or more processors. For example, a processing device may be implemented using one or more general-purpose or special purpose computers, such as, for example, a processor, an image processor, a controller and an arithmetic logic unit, a central processing unit (CPU), a graphics processing unit (GPU), a digital signal processor (DSP), a microcomputer, a field programmable array, a programmable logic unit, an application-specific integrated circuit (ASIC), a microprocessor or any other device capable of responding to and executing instructions in a defined manner.

The terms “module”, and “unit,” as used herein, may refer to, but are not limited to, a software or hardware component or device, such as a Field Programmable Gate Array (FPGA) or Application Specific Integrated Circuit (ASIC), which performs certain tasks. A module or unit may be configured to reside on an addressable storage medium and configured to execute on one or more processors. Thus, a module or unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules/units may be combined into fewer components and modules/units or further separated into additional components and modules.

Some example embodiments of the present disclosure can also be embodied as a computer readable medium including computer readable code/instruction to control at least one component of the above-described example embodiments. The medium may be any medium that can storage and/or transmission the computer readable code.

Aspects of the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The media may be transfer media such as optical lines, metal lines, or waveguides including a carrier wave for transmitting a signal designating the program command and the data construction. Examples of program instructions include both machine code, such as produced by a compiler, and files containing higher level code that may be executed by the computer using an interpreter. The described hardware devices may be configured to act as one or more software modules in order to perform the operations of the above-described example embodiments, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA). Some or all of the operations performed according to the above-described example embodiments may be performed over a wired or wireless network, or a combination thereof.

Each block of the flowchart illustrations may represent a unit, module, segment, or portion of code, which comprises one or more executable instructions for implementing the specified logical function(s). It should also be noted that in some alternative implementations, the functions noted in the blocks may occur out of the order. For example, two blocks shown in succession may in fact be executed substantially concurrently or the blocks may sometimes be executed in the reverse order, depending upon the functionality involved. Also, while an illustration may show an example of the direction of flow of information for a process, the direction of flow of information may also be performed in the opposite direction for a same process or for a different process.

Although example embodiments of the present invention have been shown and described, it would be appreciated by those skilled in the art that changes may be made to these embodiments without departing from the principles and spirit of the invention, the scope of which is defined in the claims and their equivalents.

What is claimed is:

1. A marker comprising:
a basal surface; and
a plurality of reference lines provided at the basal surface in a longitudinal direction of the basal surface, the reference lines having different gradients.

2. The marker according to claim 1, wherein the plurality of reference lines includes:
a first reference line parallel to the longitudinal direction of the basal surface; and
a second reference line having a predetermined gradient with respect to the first reference line.

3. The marker according to claim 2, wherein the basal surface, the first reference line and the second reference line have different colors respectively.

4. The marker according to claim 2,
wherein the marker has a first end and a second end in a longitudinal direction thereof, and
a distance between the first reference line and the second reference line increases from the first end to the second end of the marker.

5. The marker according to claim 1, wherein the marker has a length to surround a part of the periphery of a surgical instrument.

6. A method of estimating a surgical instrument pose using a marker, the method comprising:
detecting the marker from an image acquired via a camera; and
estimating a pose of a surgical instrument using the detected marker,
wherein the marker comprises a basal surface and a plurality of reference lines provided at the basal surface in a longitudinal direction of the basal surface, the reference lines having different gradients.

7. The method according to claim 6, wherein detecting of the marker includes:
extracting a region corresponding to the basal surface from the image;
removing noise from the extracted region;
detecting a rim of the region from which noise has been removed;
determining whether the detected rim of the region has a same shape as a rim of a preset marker; and
acquiring a color image of the extracted region if the rim of the region has the same shape as the rim of the preset marker.

8. The method according to claim 7, wherein the extracting of the region corresponding to the basal surface includes:
converting the image into a black-and-white image; and
subjecting the converted black-and-white image to binarization based on a color-brightness of the basal surface.

9. The method according to claim 8, wherein removing the noise from the extracted region includes filling an empty portion within the extracted region by performing a closing operation of the binary image.

10. The method according to claim 7, wherein, extraction of the region corresponding to the basal surface from the image is repeated if it is determined the rim of the region does not have the same shape as the rim of the preset marker.

11. The method according to claim 7, further comprising, after acquisition of the color image of the region, judging whether the plurality of reference lines is present in the acquired color image,
wherein the color image is used as the marker if the plurality of reference lines is present in the color image.

12. The method according to claim 6, wherein estimating of the pose of the surgical instrument includes:
acquiring position information on each apex of the detected marker;
marking the marker according to the position information in an X-Y plane;
extracting the plurality of reference lines from the marker;
estimating a position of the surgical instrument using a relationship between the plurality of extracted reference lines and the X-axis of the X-Y plane; and
estimating the pose of the surgical instrument using a length ratio of two sides of the marker parallel to the Y-axis of the X-Y plane as well as the relationship between the plurality of extracted reference lines and the X-axis of the X-Y plane.

13. The method according to claim 12, wherein estimating of the position of the surgical instrument includes:
calculating a half line that originates from a center of a first reference line and passes through the center of a second reference line among the plurality of extracted reference lines;
selecting a reference apex of the marker using an angle between the calculated half line and the X-axis;
matching the marker to a preset real marker using the selected reference apex; and
estimating the position of the surgical instrument using the position information on each apex of the marker and identification information on the preset real marker.

14. The method according to claim 13, wherein, upon selection of the reference apex of the marker,
an apex located closest to the reference line from which the half line originates, among apexes having positive values when substituted into a preset linear equation, is selected as the reference apex if the angle between the half line and the X-axis is 180 degrees or more, and
an apex located closest to the reference line from which the half line originates, among apexes having negative values when substituted into a preset linear equation, is selected as the reference apex if the angle between the half line and the X-axis is less than 180 degrees.

15. The method according to claim 12, wherein estimating of the pose of the surgical instrument is implemented by calculating at least one of a roll-direction rotation angle, a yaw-direction rotation angle, and a pitch-direction rotation angle of the marker.

16. The method according to claim 15, wherein the roll-direction rotation angle of the marker is calculated using a distance between centers of the plurality of reference lines.

17. The method according to claim 15, wherein the yaw-direction rotation angle of the marker is calculated using an angle between the X-axis and a half line that originates from a center of a first reference line and passes through a center of a second reference line among the plurality of reference lines.

18. The method according to claim 15, wherein the pitch-direction rotation angle of the marker is calculated using a length ratio of two sides of the marker parallel to the Y-axis of the X-Y plane.

19. The method according to claim 18, wherein, a first side among the two sides of the marker parallel to the Y-axis is closer to the Y-axis than the second side among the two sides, and
the pitch-direction rotation angle of the marker has a positive value if the length of the first side is less than the length of the second side, and the pitch-direction rotation angle of the marker has a negative value if the length of the first side is greater than the length of the second side.
